(19) United States
(12) Patent Application Publication
(10) Pub. No.: US 2026/0295712 A1
(43) Pub. Date: Oct. 1, 2026

(54) INTRAVASCULAR DEVICE INCLUDING HIGH VOLTAGE COAXIAL CONDUCTOR WIRING

(71) Applicant: Biosense Webster ( Israel) Ltd., Yokeam (IL)

(72) Inventors: Keshava DATTA, Chino Hills, CA (US); Anand RAO, Tustin, CA (US); Thanh NGUYEN, El Monte, CA (US); Thomas V. SELKEE

(73) Assignee: Biosense Webster (Israel) Ltd., Yokneam (IL)

(21) Appl. No.: 19/709,680

(22) Filed: Jun. 16, 2026

Related U.S. Application Data
(63) Continuation of application No. 18/065,709, filed on Dec. 14, 2022, now Pat. No. 12,661,737.
(60) Provisional application No. 63/301,082, filed on Jan. 20, 2022.

(51) Int. Cl. B23K 11/30; H05H 1/34

(52) U.S. Cl. CPC B23K 11/3009; H05H 1/34

(57) ABSTRACT

Wiring is disclosed herein that is configured to supply IRE ablation signals through a catheter body and is resistant against dielectric breakdown, arcing, and noise during ablation. The wiring includes a highly conductive core, a conductive cover surrounding the core that has lower electrical and/or thermal conductivity than the core, and an insulative jacket surrounding the conductive cover. A catheter including such wiring may be suitable for supplying electrical signals to tissue to perform IRE ablation. In some examples, such a catheter can also be suitable for reversible electroporation and/or RF ablation.

### CROSS REFERENCE TO RELATED APPLICATIONS

This is a continuation of, and claims priority under 35 U.S.C. § 120 to, prior filed U.S. patent application Ser. No. 18/065,709 filed Dec. 14, 2022 (Attorney Docket No. BIO6567USNP1_253757.334), which claims the benefit of priority under 35 U.S.C. § 119 to prior filed U.S. Provisional Patent Application No. 63/301,082, filed Jan. 20, 2022 (Attorney Docket Number 253757.000093 (BIO6567USPSP1)), the entire contents of which is hereby incorporated by reference as if set forth in full herein.

### FIELD

The present invention relates generally to ablation catheters, and particularly to ablation catheters having wiring suitable to withstand high voltages applied during ablation, for instance during pulsed field ablation (PFA), also referred to herein as irreversible electroporation (IRE).

### BACKGROUND

Cardiac arrhythmia, such as atrial fibrillation, occurs when regions of cardiac tissue abnormally conduct electric signals to adjacent tissue, thereby disrupting the normal cardiac cycle and causing asynchronous rhythm. Sources of undesired signals are typically located in tissue of the atria and ventricle. Regardless of source, unwanted signals are conducted elsewhere through heart tissue where they can initiate or continue arrhythmia.

Treatment of cardiac arrhythmia can include inserting an elongated catheter through patient vasculature and into a heart and applying electrical signals to ablate cardiac tissue, thereby disrupting conductive pathways of electrical signals causing arrhythmia. The ablation serves to cease or modify the propagation of unwanted electrical signals from one portion of the heart to another. Presently the most common ablation technique involves applying radio frequency (RF) electrical signals via electrodes to tissue to generate heat. Irreversible electroporation (IRE) ablation (pulse field ablation) is a more recently developed technique which involves applying short duration high voltage pulses between electrodes in a biphasic way across tissue to cause cell death.

Generally, structures for a catheter suitable for RF ablation and a catheter suitable for IRE ablation can be similar, and some catheters may be suitable for both RF and IRE ablation. However, differences in electrical signal application including waveform shape, voltage amplitude, etc. as well as geometry of electrodes at a target site may result in diverging design considerations for RF and IRE ablation catheters. Generally, IRE ablation signals include short pulses having a higher voltage amplitude (suitable to induce electric fields across cells to induce irreversible electroporation) while RF ablation signals are continuous sinusoids having a lower voltage amplitude.

Current typical ablation practices utilize RF ablation alone, with some treatments utilizing IRE ablation alone. U.S. Patent Pub. Nos. 2021/0161592, 2021/0169550, and 2021/0177503, each incorporated by reference herein and attached in the Appendix to priority application U.S. 63/301,082, each disclose a respective system suitable for both RF and IRE ablation in a single treatment and also illustrate differences between RF and IRE signals applied during ablation.

IRE ablation to treat cardiac arrhythmia typically requires voltages of about 900 V or greater when in a bipolar configuration. Voltage this high through the existing wiring can degrade the insulation which allows for more arcing. Given the size and biocompatibility constraints of the devices used to treat cardiac arrhythmia the positioning and dimensions of the wiring cannot be altered to compensate for these effects. Thus, new materials are needed for the wiring.

### SUMMARY

Examples presented herein generally include wiring configured to supply IRE ablation signals through a catheter body that are resistant against dielectric breakdown, arcing, and noise during ablation. The wiring includes a highly conductive core, a conductive cover surrounding the core that has lower electrical and/or thermal conductivity than the core, and an insulative jacket surrounding the conductive cover. An intravascular device (e.g. catheter) including such wiring may be suitable for supplying electrical signals to tissue to perform IRE ablation. In some examples, such an intravascular device can also be suitable for reversible electroporation and/or RF ablation.

A first example intravascular device can include an elongated shaft, a distal section, and a plurality of wires. The elongated shaft can extend along a longitudinal axis. The elongated shaft can be sized to traverse vasculature. The distal section can include a plurality of electrodes configured to deliver at least 900 Volts for irreversible electroporation. The plurality of wires each can be electrically joined to a respective electrode of the plurality of electrodes. Each of the wires can extend through at least a portion of the elongated shaft. Each of the wires can respectively include an electrically conductive core material having a first electrical conductivity and an electrically conductive cover material circumscribing the electrically conductive core material and having a second electrical conductivity less than the first electrical conductivity. Each of the wires can also respectively include an insulative jacket circumscribing the electrically conductive cover material such that no electrical arc is generated between the plurality of wires during delivery of at least 900 Volts to each electrode.

The electrically conductive core material can include about 12% to about 50% of silver.

The electrically conductive core material can include about 28% to about 33% of silver.

The electrically conductive core material can include about 12% to about 50% of copper.

The first electrical conductivity (of the core material) can measure about 4×10⁷ S/m to about 6×10⁷ S/m when measured at 20° C.

The second electrical conductivity (of the cover material) can measure about 1×10⁶ S/m when measured at 20° C.

The first electrical conductivity can measure at least ten times greater than the second electrical conductivity when measured at 20° C.

The electrically conductive core material can have a first thermal conductivity, and the electrically conductive cover material can have a second thermal conductivity less than the first thermal conductivity.

The first thermal conductivity can be about 300 Watts per millikelvin (W/mK) to about 400 W/mK.

The second thermal conductivity can be about 11.2 W/mK.

The first thermal conductivity can be at least ten times the second thermal conductivity.

Each of the wires can have a resistance per length of wire measuring about 1.4 Ohms per foot (Ω/ft) (4.6 Ω/m) to about 1.8 Ω/ft (5.8 Ω/m).

Each of the wires of can have a wire resistance of about 10 Ohms (Ω) to about 12 Ω.

The electrically conductive cover material of each wire can have a diameter measuring between about 0.0031 inches (79 micrometers or microns) to about 0.0040 inches (102 micrometers).

A second example intravascular device can include an elongated shaft, a distal section, and a plurality of wires. The elongated shaft can extend along a longitudinal axis and can be sized to traverse vasculature. The distal section can include a plurality of electrodes. The wires can each be electrically joined to a respective electrode of the plurality of electrodes. Each of the wires can extend through at least a portion of the elongated shaft. Each of the wires can respectively include a plurality of strands, each strand respectively including an electrically conductive core material having a first electrical conductivity and an electrically conductive cover material circumscribing the core material and having a second electrical conductivity less than the first electrical conductivity. Each wire can also include an insulative jacket circumscribing the plurality of strands so that no electrical arc is generated between the plurality of wires when at least 900 Volts is provided to each electrode.

The electrically conductive core material of each of the strands can include about 41% silver (Ag).

Each strand can have a diameter measuring about 0.002 inches (51 micrometers).

At least a portion of the wires can have exactly four (4) strands.

At least a portion of the strands can have a resistance of about 20 Ω.

Each of the wires can be configured to withstand bipolar pulses resulting in a voltage difference of about 1,500 volts to about 2,000 volts between two wires.

Each of the wires can be configured to withstand bipolar pulses resulting in a voltage difference of about 1,500 volts between two wires in the plurality of wires.

Each of the wires can be configured to withstand bipolar pulses resulting in a voltage difference of about 1,800 volts between two wires in the plurality of wires.

The first example intravascular device and/or the second intravascular device can be configured such that the distal section includes a lasso region upon which the plurality of electrodes are mounted. The lasso region can be configured to move between an elongated configuration aligned to the longitudinal axis to a circular shape generally orthogonal to the longitudinal axis.

The first example intravascular device and/or the second intravascular device can be configured such that the distal section includes a plurality of spines upon which the plurality of electrodes are mounted. The spines can be configured to move from an elongated configuration aligned to the longitudinal axis such that the spines bow outwardly from the longitudinal axis to form an expanded form.

The first example intravascular device and/or the second intravascular device can be configured such that the plurality of wires includes 12 to 8 wires.

The first example intravascular device and/or the second intravascular device can be configured such that the elongated shaft can have a diameter of about 8.5 French (2.8 millimeters).

The first example intravascular device and/or the second intravascular device can be configured such that the plurality of wires are bundled in an insulative sleeve.

The first example intravascular device and/or the second intravascular device can be configured such that each wire of the plurality of wires comprising a diameter measuring about 0.0053 inches (130 micrometers).

The first example intravascular device and/or the second intravascular device can be configured such that the insulative jacket has a wall thickness measuring about 0.0008 inches (20 micrometers) to about 0.00125 inches (32 micrometers).

The first example intravascular device and/or the second intravascular device can be configured such that the electrically conductive cover material includes about 33% to about 37% nickel, about 30% to about 38% cobalt, about 19% to about 21% chromium, and about 9% to about 10.5% molybdenum.

The first example intravascular device and/or the second intravascular device can be configured such that the electrically conductive cover material further includes about 1% iron.

The first example intravascular device and/or the second intravascular device can be configured such that the electrically conductive cover material further includes less than 1% of at least one element of a group of elements consisting of: carbon, manganese, silicon, phosphorus, sulfur, titanium, and boron.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example solid core wire according to aspects of the present invention.

FIG. 2 is an illustration of an example wire bundle of solid core wires according to aspects of the present invention.

FIG. 3 is an illustration of an example stranded wire according to aspects of the present invention.

FIG. 4 is an illustration of an example wire bundle of stranded wires according to aspects of the present invention.

FIG. 5 is an illustration of a profile view of an example catheter according at aspects of the present invention.

FIG. 6 is an illustration of a cross-sectional view of the example catheter as indicated in FIG. 5.

FIG. 7 is an illustration of an end view of the example catheter as indicated in FIG. 5.

FIG. 8 is an illustration of another example catheter according to aspects of the present invention.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

As discussed herein, vasculature of a “patient,” “host,” “user,” and “subject” can be vasculature of a human or any animal. It should be appreciated that an animal can be a variety of any applicable type, including, but not limited thereto, mammal, veterinarian animal, livestock animal or pet type animal, etc. As an example, the animal can be a laboratory animal specifically selected to have certain characteristics similar to a human (e.g., rat, dog, pig, monkey, or the like). It should be appreciated that the subject can be any applicable human patient, for example.

As discussed herein, “operator” and “user” can include a doctor, surgeon, technician, scientist, or any other individual or delivery instrumentation associated with delivery of a multi-electrode catheter for treatments disclosed herein.

As discussed herein, the term “ablate” or “ablation”, as it relates to the devices and corresponding systems of this disclosure, refers to components and structural features configured to reduce or prevent the generation of erratic cardiac signals in the cells by utilizing non-thermal energy, such as irreversible electroporation (IRE), referred throughout this disclosure interchangeably as pulsed field ablation (PFA). Ablating or ablation as it relates to the devices and corresponding systems of this disclosure is used throughout this disclosure in reference to ablation of cardiac tissue for certain conditions including, but not limited to, arrhythmias, atrial flutter ablation, pulmonary vein isolation, supraventricular tachycardia ablation, and ventricular tachycardia ablation. The term “ablate” or “ablation” also includes known methods, devices, and systems to achieve various forms of bodily tissue ablation as understood by a person skilled in the relevant art.

As discussed herein, the terms “bipolar” and “unipolar” when used to refer to IRE ablation schemes describe ablation schemes which differ with respect to electrical current path and electric field distribution. “Bipolar” refers to an IRE ablation scheme utilizing a current path between two electrodes that are both positioned at a treatment site; current density and electric flux density is typically approximately equal at each of the two electrodes. “Unipolar” refers to an IRE ablation scheme utilizing a current path between two electrodes where one electrode having a high current density and high electric flux density is positioned at a treatment site, and a second electrode having comparatively lower current density and lower electric flux density is positioned remotely from the treatment site.

As discussed herein, the terms “biphasic pulse” and “monophasic pulse” refer to respective electrical signals. “Biphasic pulse” refers to an electrical signal having a positive-voltage phase pulse (referred to herein as “positive phase”) and a negative-voltage phase pulse (referred to herein as “negative phase”). “Monophasic pulse” refers to an electrical signal having only a positive or only a negative phase. Preferably, a system providing the biphasic pulse is configured to prevent application of a direct current voltage (DC) to a patient. For instance, the average voltage of the biphasic pulse can be zero volts with respect to ground or other common reference voltage. Additionally, or alternatively, the system can include a capacitor or other protective component. Where voltage amplitude of the biphasic and/or monophasic pulse is described herein, it is understood that the expressed voltage amplitude is an absolute value of the approximate peak amplitude of each of the positive-voltage phase and/or the negative-voltage phase. Each phase of the biphasic and monophasic pulse preferably has a square shape having an essentially constant voltage amplitude during a majority of the phase duration. Phases of the biphasic pulse are separated in time by an interphase delay. The interphase delay duration is preferably less than or approximately equal to the duration of a phase of the biphasic pulse. The interphase delay duration is more preferably about 25% of the duration of the phase of the biphasic pulse.

As discussed herein, the terms “tubular” and “tube” are to be construed broadly and are not limited to a structure that is a right cylinder or strictly circumferential in cross-section or of a uniform cross-section throughout its length. For example, the tubular structures are generally illustrated as a substantially right cylindrical structure. However, the tubular structures may have a tapered or curved outer surface without departing from the scope of the present disclosure.

The present disclosure is related to systems, method or uses and devices for IRE ablation of cardiac tissue to treat cardiac arrhythmias. Ablative energies can be provided to cardiac tissue by a tip portion of a catheter which can deliver ablative energy to the tissue to be ablated. Some example catheters include three-dimensional structures at the tip portion and are configured to administer ablative energy from various electrodes positioned on the three-dimensional structures. Ablative procedures incorporating such example catheters can be visualized using fluoroscopy.

Ablation of cardiac tissue using application of a thermal technique, such as radio frequency (RF) energy and cryoablation, to correct a malfunctioning heart is a well-known procedure. Typically, to successfully ablate using a thermal technique, cardiac electropotentials need to be measured at various locations of the myocardium. In addition, temperature measurements during ablation provide data enabling the efficacy of the ablation. Typically, for an ablation procedure using a thermal technique, the electropotentials and the temperatures are measured before, during, and after the actual ablation. RF approaches can have risks that can lead to tissue charring, burning, steam pop, phrenic nerve palsy, pulmonary vein stenosis, and esophageal fistula. Cryoablation is an alternative approach to RF ablation that can reduce some thermal risks associated with RF ablation. However maneuvering cryoablation devices and selectively applying cryoablation is generally more challenging compared to RF ablation; therefore, cryoablation is not viable in certain anatomical geometries which may be reached by electrical ablation devices.

While RF ablation and cryoablation are based on thermal energy transfer to induce local tissue necrosis, the solution of this disclosure resolves these and other problems by utilizing irreversible electroporation (IRE). IRE as discussed in this disclosure is a non-thermal cell death technology that can be used for ablation of atrial arrhythmias. To ablate using IRE/PEF, biphasic voltage pulses are applied to disrupt cellular structures of myocardium. The biphasic pulses are non-sinusoidal and can be tuned to target cells based on electrophysiology of the cells. In contrast, to ablate using RF, a sinusoidal voltage waveform is applied to produce heat at the treatment area, indiscriminately heating all cells in the treatment area. IRE therefore has the capability to spare adjacent heat sensitive structures or tissues which would be of benefit in the reduction of possible complications known with ablation or isolation modalities. Additionally, or alternatively, monophasic pulses can be utilized.

Electroporation can be induced by applying a pulsed electric field across biological cells to cause reversable (temporary) or irreversible (permanent) creation of pores in the cell membrane. Reversible electroporation utilizes pulsed electrical signals similar to IRE, however electricity applied with the electrodes is below the electric field threshold of the target tissue allowing cells to repair. The cells have a transmembrane electrostatic potential that is increased above a resting potential upon application of the pulsed electric field. While the transmembrane electrostatic potential remains below a threshold potential, the electroporation is reversable, meaning the pores can close when the applied pulse electric field is removed, and the cells can self-repair and survive. If the transmembrane electrostatic potential increases beyond the threshold potential, the electroporation is irreversible, and the cells become permanently permeable. As a result, the cells die due to a loss of homeostasis and typically die by apoptosis. Generally, cells of differing types have differing threshold potential. For instance, heart cells have a threshold potential of approximately 500 V/cm, whereas for bone it is 3000 V/cm. These differences in threshold potential allow IRE to selectively target tissue based on threshold potential.

The solution of this disclosure includes systems and methods for applying electrical signals from catheter electrodes positioned in the vicinity of myocardial tissue to generate a pulsed electric field effective to induce electroporation in the myocardial tissue. The systems and methods can be effective to ablate targeted tissue by inducing irreversible electroporation. In some examples, the systems and methods can be effective to induce reversible electroporation as part of a diagnostic procedure. Reversible electroporation can temporarily interrupt electrical activation signals through targeted myocardial tissue to allow for observation of changes in electrical activity within the heart which can be used to predict the outcome of irreversible electroporation at the targeted tissue. Temporarily interrupted electrical activation signals induced through reversible electroporation can resume as the cells self-repair.

The pulsed electric field, and its effectiveness to induce reversible and/or irreversible electroporation, can be affected by physical parameters of the system and biphasic pulse parameters of the electrical signal. Physical parameters can include electrode contact area, electrode spacing, electrode geometry, etc. Examples presented herein generally include physical parameters adapted to effectively induce reversible and/or irreversible electroporation. Biphasic pulse parameters of the electrical signal can include voltage amplitude, pulse duration, pulse interphase delay, inter-pulse delay, total application time, delivered energy, etc. In some examples, parameters of the electrical signal can be adjusted to induce both reversible and irreversible electroporation given the same physical parameters. Examples of various systems and methods of ablation including IRE are presented in U.S. Patent Pub. Nos. 2021/0169550, 2021/0169567, 2021/0169568, 2021/0161592, 2021/0196372, 2021/0177503, and 2021/0186604 the entireties of each of which are incorporated herein by reference and attached in the Appendix to priority application U.S. 63/301,082. Example systems and methods for reversible electroporation are disclosed in U.S. Patent Pub. No. 2021/0162210 the entirety of which is incorporated herein by reference and attached in the Appendix to priority application U.S. 63/301,082.

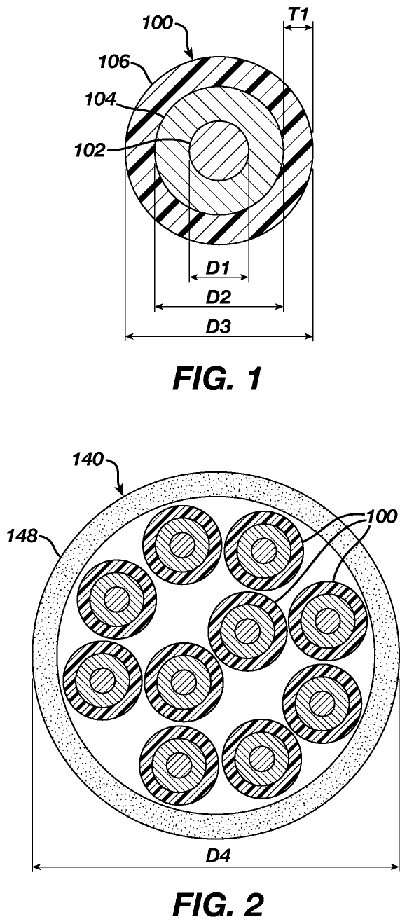

FIG. 1 is an illustration of an example solid core wire 100 having a dual layer conductor including an electrically conductive core 102, an electrically conductive cover 104. The solid core wire 100 further includes an insulative jacket 106 over the conductive cover 104. The conductive core 102 can have a higher electrical conductivity than the conductive cover 104 to reduce likelihood of arcing or ionization between adjacent wires and/or reduce electrical signal noise in a catheter in which the wires are configured to deliver electrical energy the electrodes for IRE ablation. IRE ablation for atrial fibrillation can require voltage of between about 900 Volts and higher than 2,500 Volts between electrodes in bipolar mode, resulting in a corresponding voltage difference between wires in the catheter. In comparison, voltages applied during RF ablation can typically range between 10 to 200 Volts. The higher voltage, shorter duration electrical pulses applied during IRE ablation can present greater likelihood of noise, ionization, and/or arcing between wires compared to lower voltage sinusoidal signals applied during RF ablation. The example wire 100 can mitigate noise, ionization, and/or arcing in some example catheters compared to known typical solid core wire having a uniform conductor material.

In some examples, the solid core wire 100 can be configured such that no electrical arc is generated between wires during delivery of at least 900 Volts to each electrode. In some examples the wire 100 can be configured such that no electrical arc is generated between wires during delivery of between about 1,500 Volts and about 2,000 Volts. In some examples the wire 100 can be configured such that no electrical arc is generated between wires during delivery of about 1,800 Volts.

The conductive core 102 preferably has a high electrical conductivity to result in a higher current density in the conductive core 102 compared to the conductive cover 104. To that end, the conductive core 102 can include highly conductive material such as copper or silver. In some examples, the material of the conductive core 102 can include about 12% to about 50% of silver and/or copper. For instance, the electrically conductive core material can include about 12% to about 50% of silver, preferably about 28% to about 33% of silver. In some examples, the material of the conductive core 102 can include about 12% to about 50% of copper. Alternatively, the conductive core 102 can include materials not listed here to achieve similar electrical and/or thermal conductivity to the material compositions of the conductive core 102 that are disclosed herein as understood by a person skilled in the pertinent art.

The material of the electrically conductive cover 104 can include a material having a lower electrical conductivity compared to the conductive core 102. The material of the conductive cover 104 can include about 33% to about 37% nickel, about 30% to about 38% cobalt, about 19% to about 21% chromium, and about 9% to about 10.5% molybdenum, similar to 35N LT® wire by Fort Wayne Metals in Fort Wayne Indiana, USA. Alternatively, the conductive cover 104 can include materials not listed here to achieve similar electrical and/or thermal conductivity to the material compositions of conductive cover 104 that are disclosed herein as understood by a person skilled in the pertinent art.

The electrical conductivity of the material of the conductive core 102 can measure about 4×10⁷ S/m to about 6×10⁷ S/m when measured at 20° C.

The electrical conductivity of the material of the conductive cover 104 can measure about 1×10⁶ S/m when measured at 20° C.

The electrical conductivity of the material of the core 102 can measure at least ten times greater than the electrical conductivity of the material of the cover 104 when measured at 20° C.

The material of the conductive core 102 can have a thermal conductivity that is greater than thermal conductivity of the material of the conductive cover 104.

The material of the conductive core 102 can have thermal conductivity of about 300 W/mK to about 400 W/mK. The material of the conductive cover 104 can have a thermal conductivity can be about 11.2 W/mK.

The thermal conductivity of the material of the conductive core 102 can be at least ten times the thermal conductivity of the material of the conductive cover 104.

The wire 100 can have a resistance per length of wire measuring about 1.4 Ω/ft (4.6 Ω/m) to about 1.8 Ω/ft (5.8 Ω/m).

The cover 104 can have a diameter D2 measuring between about 0.0031 inches (79 micrometers) to about 0.0040 inches (102 micrometers).

The insulative jacket 106 can define a diameter D3 of the wire that measures about 0.0053 inches (130 micrometers).

The insulative jacket 106 can have a wall thickness T1 measuring about 0.0008 inches (20 micrometers) to about 0.00125 inches (32 micrometers).

FIG. 2 is an illustration of an example wire bundle 140 of solid core wires 100. The wire bundle 140 can include an insulative sleeve 148 that surrounds the wires 100. Each wire 100 in the wire bundle 140 can be connected to an electrode configured to deliver electrical energy for ablation, preferably IRE ablation, therefore the wire bundle 140 can include a number of wires equal to the number of ablation electrodes of the catheter. The wire bundle 140 can include eight (8) to twelve (12) wires 100, preferably ten (10) wires as illustrated.

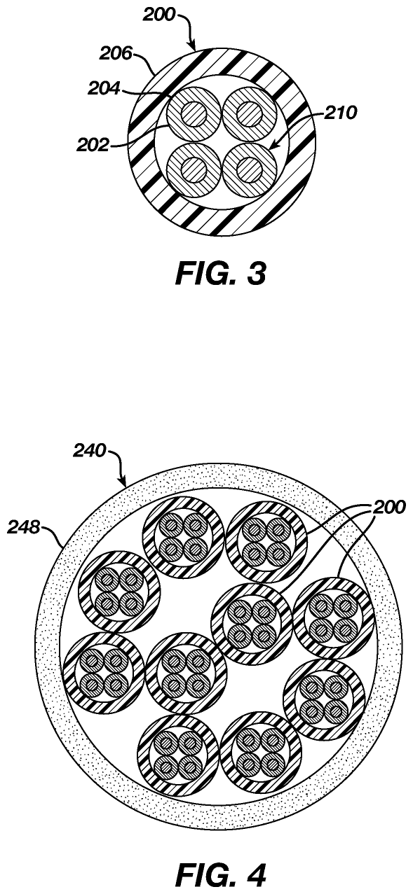

FIG. 3 is an illustration of an example stranded wire 200. The stranded wire 200 can include a number of strands 210. Each strand 210 can include a dual layer conductor including an electrically conductive core 202 and an electrically conductive cover 204. The stranded wire 200 further includes an insulative jacket 206 over the strands 210. The conductive core 202 can have a higher electrical conductivity than the conductive cover 204 to reduce likelihood of arcing or ionization between adjacent wires and/or reduce electrical signal noise in a catheter in which the wires are configured to deliver electrical energy the electrodes for IRE ablation similar to as described in relation to the solid core wire 100 illustrated in FIG. 1. The conductive core 202 can have similar material properties (e.g., electrical conductivity and thermal conductivity) as the conductive core 102 of the solid core wire 100 illustrated in FIG. 1. The conductive core 202 can have similar material composition as the conductive core 102 of the sold core wire 100 illustrated in FIG. 1. The conductive cover 204 can have similar material properties (e.g., electrical conductivity and thermal conductivity) as the conductive cover 104 of the solid core wire 100 illustrated in FIG. 1. The conductive cover 204 can have similar material composition as the conductive cover 104 of the sold core wire 100 illustrated in FIG. 1.

In some examples, the stranded wire 200 can be configured such that no electrical arc is generated between wires during delivery of at least 900 Volts to each electrode. In some examples the wire 200 can be configured such that no electrical arc is generated between wires during delivery of between about 1,500 Volts and about 2,000 Volts. In some examples the wire 200 can be configured such that no electrical arc is generated between wires during delivery of about 1,800 Volts.

FIG. 4 is an illustration of an example wire bundle 240 of stranded wires 200. The wire bundle 240 can include an insulative sleeve 248 that surrounds the wires 200. Each wire 200 in the wire bundle 240 can be connected to an electrode configured to deliver electrical energy for ablation, preferably IRE ablation, therefore the wire bundle 240 can include a number of wires equal to the number of ablation electrodes of the catheter. The wire bundle 240 can include eight (8) to twelve (12) wires, preferably ten (10) wires 200 as illustrated.

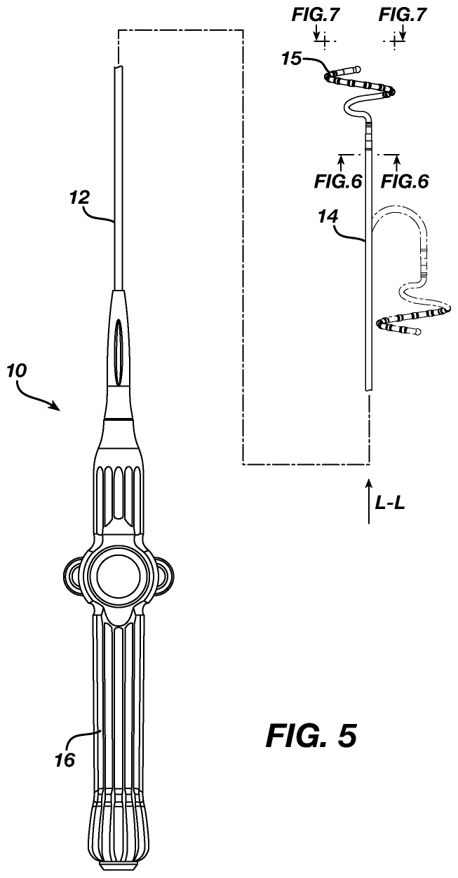

FIG. 5 is an illustration of a profile view of an example catheter 10 that can include a plurality of wires including example solid core wires 100 and/or example stranded wires 200. The catheter 10 includes a distal section 15 having a circular region that is generally traverse to a longitudinal axis L-L defined by an elongated shaft 12 of the catheter 10. The catheter 10 includes an intermediate section 14 extended along the longitudinal axis L-L distal from the elongated shaft 12. The intermediate section 14 can deflect from the longitudinal axis L-L in response to manipulation of a handle 16 at a proximal end of the catheter 10. As illustrated, the intermediate section 14 can be capable of bending approximately 180°.

In an example treatment, a suitable guiding sheath is inserted into the patient with its distal end positioned at a desired treatment location. An example of a suitable guiding sheath for use with the example catheter 10 is the Vizigo™ Braiding Guiding Sheath, commercially available from Biosense Webster, Inc. (California, USA). The distal end of the sheath is guided into one of the atria. As the catheter 10 is fed through the guiding sheath, the distal section 15 is straightened to fit through the sheath. Once the distal end of the catheter is positioned at the desired treatment location, the guiding sheath is pulled proximally, allowing the intermediate section 14 and the distal section 15 to extend outside the sheath, and the distal section 15 is free to move to its circular shape. The distal section is then inserted into a pulmonary vein or other tubular region (such as the coronary sinus, superior vena cava, or inferior vena cava) so that the outer circumference of the distal section 15 is in contact with a circumference inside the tubular region.

Wires 100, 200 in the catheter 10 can have a length of about 7 ft, or 2 m. Each of the wires 100, 200 of can have a wire resistance of about 10 Ω to about 12 Ω.

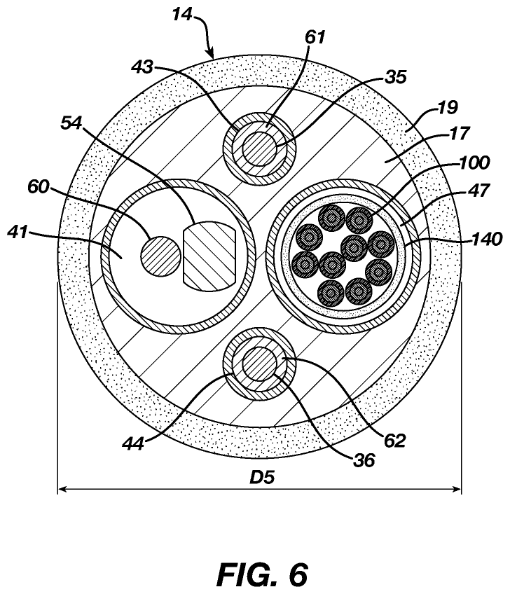

FIG. 6 is an illustration of a cross-sectional view of the example catheter 10 as indicated in FIG. 5. The intermediate section 14 includes an inner, intermediate tube 17 having four lumens 41, 43, 44, 47 therethrough. The intermediate tube 17 can be modified to include alternative numbers of lumens as understood by a person skilled in the pertinent art according to the teachings herein. The catheter 10 includes a support member 54 having a proximal end affixed within a first lumen 41 of the intermediate section 14. The support member 54 can extend into the distal section 15 of the catheter to form the distal section 15 into the circular shape. The catheter 10 includes a first pull wire/contraction wire 35 extending from the handle 16, through the shaft 12, through a second lumen 43 within the intermediate tube 17. Manipulation of the handle 16 can cause tension in the contraction wire 35 to cause the distal section 15 to contract in diameter. Within the second lumen 43, the catheter 10 can include a compression coil 61 circumscribing the contraction wire 35.

The catheter 10 includes a second pull wire/deflection wire 36 extending from the control handle 16, through the shaft, and through at least a portion of a third lumen 44 of the intermediate tube 17. The intermediate section 14 can be configured to deflect from the longitudinal axis L-L in response to manipulation of the deflection wire 36 by the control handle 16. Within the third lumen 44, the catheter 10 can include a compression coil 62 circumscribing the deflection wire 36.

The catheter 10 can include a navigation sensor assembly 60 that extends from the handle 16 through the first lumen 41 in the intermediate tube 17 and into the distal section 15.

The catheter 10 can include lead wires 100 extending proximally from electrodes 26 on the distal region 15, through a fourth lumen 47 through the intermediate tube 17, through the shaft 12, through the control handle 16, and terminate at their proximal end in a connector (not shown) which is connected to an appropriate system configured to receive electrical signals for mapping and/or transmit energy for ablation. The lead wires 100 can be attached to the electrodes 26 of the distal region 15 by any compatible conventional technique. The lead wires 100 can be individually insulated and bundled within the insulating sleeve 148 to form the wire bundle 140. The lead wires can be solid core wires 100 as illustrated and/or stranded wires 200.

The catheter 10 can include an outer tube 19 circumscribing the intermediate tube 17 and configured to provide structural stability to the intermediate section 14.

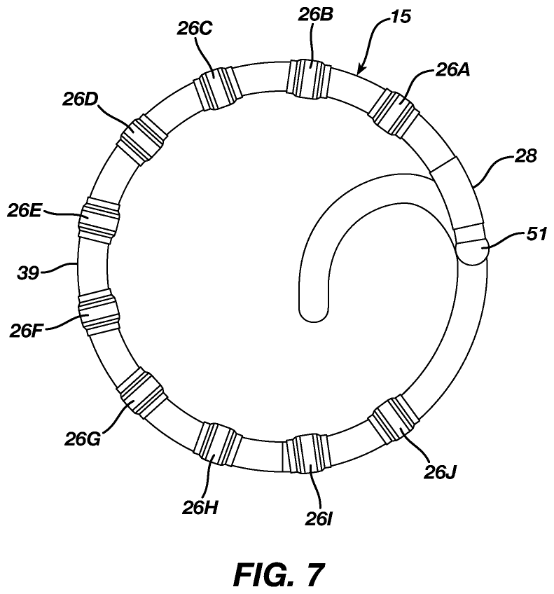

FIG. 7 is an illustration of a distal end view of the example catheter 10 as indicated in FIG. 5. The generally circular distal region 15 can curve in a clockwise direction or a counterclockwise direction. The distal region 15 can contract in a radial direction. When uncontracted, the distal region 15 can have an outer diameter preferably ranging from about 25 mm to about 35 mm. The distal region 15 can have an outer diameter when contracted preferably ranging from about 15 mm to about 25 mm.

The catheter 10 is preferably configured to provide IRE ablation voltage pulses from the electrodes 26A-J. IRE is a predominantly non-thermal process, which causes an increase of the tissue temperature by, at most, a few degrees for a few milliseconds. It thus differs from RF (radio frequency) ablation, which raises the tissue temperature by between 20 and 70° C. and destroys cells through heating. IRE can utilize monophasic pulses or biphasic pulses. The IRE pulses, alone or in combination with RF ablation, can be generated and applied in various treatments such as described in references attached in the Appendix to priority application U.S. 63/301,082.

In one example treatment, voltage pulses can be applied in a triplet sequence to perform IRE ablation. In a first triplet of the triplet sequence, a biphasic pulse can be applied between a first pair of adjacent electrodes 26A, 26B, next a biphasic pulse of similar amplitude can be applied between a second pair of adjacent electrodes 26B, 26C, and next a biphasic pulse of about twice the amplitude of the previous biphasic pulses can be applied between alternate electrodes 26A, 26C from the previous two pairs of adjacent electrodes. The triplet sequence can continue with a second triplet that includes two adjacent electrodes 26B, 26C from the first triplet and a new adjacent electrode 26D. The second triplet can follow a similar pattern of the first triplet with a biphasic pulse between a first pair of adjacent electrodes 26B, 26C, next a biphasic pulse of similar amplitude between a second pair of adjacent electrodes 26C, 26D, and next a biphasic pulse of about twice the amplitude between alternate electrodes 26B, 26D. The triplet sequence can continue with a third triplet with the next three electrodes 26C, 26D, 26E, a fourth triplet with the next three electrodes 26D, 26E, 26F, a fifth triplet with the next three electrodes 26E, 26F, 26G, a sixth triplet with the next three electrodes 26F, 26G, 26H, a seventh triplet with the next three electrodes 26G, 26H, 26I, and an eighth triplet with the next three electrodes 26H, 26I, 26J. The triplet pattern can repeat, starting again at the first triplet. The catheter 10 is preferably configured to withstand biphasic pulses between adjacent electrodes having an amplitude of about 900 Volts and biphasic pulses between alternate electrodes having an amplitude of about 1,800 Volts. Alternatively, the catheter 10 can be configured to withstand biphasic and/or monophasic pulses suitable for IRE and having a voltage amplitude as disclosed elsewhere herein and/or in the Appendix to priority application U.S. 63/301,082.

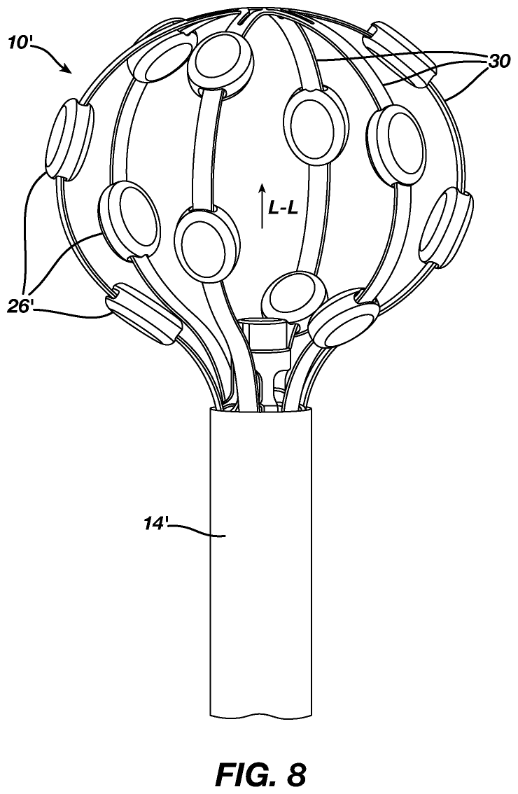

FIG. 8 is an illustration of another example catheter 10′ configured for IRE ablation and having electrodes 26′ disposed on a plurality of spines 30. The catheter 10′ includes wires 100, 200 to each of the electrodes 26′ that extend through a shaft 14'. The catheter 10′ includes twelve (12) electrodes 26′, and therefore twelve (12) wires 100, 200.

As illustrated, the spines 30 are expanded from the longitudinal axis L-L to form a basket shape that is approximately spherical. The spines 30 can be compressed to the longitudinal axis L-L to an elongated configuration that is sized to traverse vasculature. The electrodes 26′ can be positioned over the spines 30 as illustrated or otherwise attached to the spines 30 as understood by a person skilled in the pertinent art.

The catheter 10′ is preferably configured to provide IRE ablation voltage pulses between pairs of electrodes 26′. The IRE pulses, alone or in combination with RF ablation, can be generated and applied in various treatments such as described in references attached in the Appendix to priority application U.S. 63/301,082.

The present disclosure also includes additional example catheters each including example wire(s) 100, 200 as disclosed herein. For instance, example catheters can include features disclosed in U.S. Pat. Nos. 5,718,241, 6,198,974, 6,484,118, 6,987,995, 7,142,903, 7,274,957, 7,377,906, 7,591,799, 7,593,760, 7,720,517, 7,853,302, 8,000,765, 8,021,327, 8,275,440, and 11,071,585, the entireties of each of which are incorporated herein by reference and attached in the Appendix to priority application U.S. 63/301,082. The catheters disclosed in the Appendix to priority application U.S. 63/301,082 can be modified to include wire(s) 100, 200 to ablation electrodes as understood by a person skilled in the pertinent art.

The present disclosure also includes additional example systems and methods for reversible and/or irreversible electroporation modified to utilize the example catheters 10, 10′ illustrated herein and/or modified catheters as disclosed in the preceding paragraph as understood by a person skilled in the pertinent art. For instance, example systems and method can include features disclosed in U.S. Patent Pub. Nos. 2021/0169550, 2021/0169567, 2021/0169568, 2021/0161592, 2021/0196372, 2021/0177503, 2021/0186604, and 2021/0162210, the entireties of each of which are incorporated herein by reference and attached in the Appendix to priority application U.S. 63/301,082. Further, the catheters disclosed in these publications can be modified to include wire(s) 100, 200 to ablation electrodes as understood by a person skilled in the pertinent art.

The devices and/or methods of this disclosure reduced and/or eliminated arcing and noise during the application of IRE currents. The specific configurations, choice of materials and the size and shape of various elements can be varied according to design specifications or constraints. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:
1. An intravascular device comprising:
   an elongated shaft extending along a longitudinal axis and sized to traverse vasculature;
   a distal section comprising a plurality of electrodes configured to deliver at least 900VDC for irreversible electroporation; and
   a plurality of wires each electrically joined to a respective electrode of the plurality of electrodes, each of the wires of the plurality of wires extending through at least a portion of the elongated shaft, and each of the wires of the plurality of wires respectively comprising:
   an electrically conductive core material comprising a first electrical conductivity,
   an electrically conductive cover material comprising a second electrical conductivity less than the first electrical conductivity, the electrically conductive cover material circumscribing the electrically conductive core material, and
   an insulative jacket circumscribing the electrically conductive cover material such that no electrical arc is generated between the plurality of wires during delivery of at least 900 Volts to each electrode.
2. The intravascular device of clause 1, the electrically conductive core material comprising about 12% to about 50% of silver.
3. The intravascular device of clause 2, the electrically conductive core material comprising about 28% to about 33% of silver.
4. The intravascular device of any one of clauses 1-3, the electrically conductive core material comprising about 12% to about 50% of copper.
5. The intravascular device of any one of clauses 1-4, the first electrical conductivity being about 4×10⁷ S/m to about 6×10⁷ S/m when measured at 20° C.
6. The intravascular device of any one of clauses 1-5, the second electrical conductivity being about 1×10⁶ S/m when measured at 20° C.
7. The intravascular device of any one of clauses 1-6, the first electrical conductivity being at least ten times greater than the second electrical conductivity when measured at 20° C.
8. The intravascular device of any one of clauses 1-7,
   the electrically conductive core material comprising a first thermal conductivity, and
   the electrically conductive cover material comprising a second thermal conductivity less than the first thermal conductivity.
9. The intravascular device of clause 8, the first thermal conductivity being about 300 W/mK to about 400 W/mK.
10. The intravascular device of clause 8 or 9, the second thermal conductivity being about 11.2 W/mK.
11. The intravascular device of any one of clauses 8-10, the first thermal conductivity being at least ten times the second thermal conductivity.
12. The intravascular device of any one of clauses 1-11, each of the wires of the plurality of wires comprising a resistance per length of wire measuring about 1.4 Ω/ft (4.6 Ω/m) to about 1.8 Ω/ft (5.8 Ω/m).
13. The intravascular device of any one of clauses 1-12, each of the wires of the plurality of wires comprising a wire resistance of about 10 Ω to about 12 Ω.
14. The intravascular device of any one of clauses 1-13, each of the electrically conductive cover material of the plurality of wires comprising a diameter measuring between about 0.0031 inches (79 micrometers) to about 0.0040 inches (102 micrometers).
15. An intravascular device comprising:
   an elongated shaft extending along a longitudinal axis and sized to traverse vasculature;
   a distal section comprising a plurality of electrodes; and a plurality of wires each electrically joined to a respective electrode of the plurality of electrodes, each of the wires of the plurality of wires extending through at least a portion of the elongated shaft, and each of the wires of the plurality of wires respectively comprising:
   a plurality of strands, each strand respectively comprising an electrically conductive core material comprising a first electrical conductivity and an electrically conductive cover material comprising a second electrical conductivity less than the first electrical conductivity, the electrically conductive cover material circumscribing the electrically conductive core material, and an insulative jacket circumscribing the plurality of strands so that no electrical arc is generated between the plurality of wires when at least 900 Volts is provided to each electrode.
16. The intravascular device of clause 15, the electrically conductive core material of each of the strands comprising about 41% silver (Ag).
17. The intravascular device of clause 15 or 16, each strand comprising a diameter measuring about 0.002 inches (51 micrometers).
18. The intravascular device of any one of clauses 15-17, at least a portion of the wires of the plurality of wires comprising exactly four (4) strands.
19. The intravascular device of any one of clauses 15-18, at least a portion of the strands of the plurality of strands comprising a resistance of about 20 Ω.
20. The intravascular device of any one of clauses 1-19, each of the plurality of wires being configured to withstand bipolar pulses resulting in a voltage difference of about 1,500 volts to about 2,000 volts between two wires in the plurality of wires.
21. The intravascular device of clause 20, each of the plurality of wires being configured to withstand bipolar pulses resulting in a voltage difference of about 1,500 volts between two wires in the plurality of wires.
22. The intravascular device of clause 20, each of the plurality of wires being configured to withstand bipolar pulses resulting in a voltage difference that is between 1,800 volts and 2,000 volts between two wires in the plurality of wires.
23. The intravascular device of any one of clauses 1-22, the distal section comprising a lasso region upon which the plurality of electrodes are mounted, the lasso region being configured to move between an elongated configuration aligned to the longitudinal axis to a circular shape generally orthogonal to the longitudinal axis.
24. The intravascular device of any one of clauses 1-22, the distal section comprising a plurality of spines upon which the plurality of electrodes are mounted, the spines being configured to move from an elongated configuration aligned to the longitudinal axis such that the spines bow outwardly from the longitudinal axis to form an expanded form.
25. The intravascular device of any one of clauses 1-24, the plurality of wires comprising 12 to 8 wires.
26. The intravascular device of any one of clauses 1-25, the elongated shaft having a diameter of about 8.5 French (2.8 millimeters).
27. The intravascular device of any one of clauses 1-26, the plurality of wires being bundled in an insulative sleeve.
28. The intravascular device of any one of clauses 1-27, each wire of the plurality of wires comprising a diameter measuring about 0.0053 inches (130 micrometers).
29. The intravascular device of any one of clauses 1-28, the insulative jacket comprising a wall thickness measuring about 0.0008 inches (20 micrometers) to about 0.00125 inches (32 micrometers).
30. The intravascular device of any one of clauses 1-29, the electrically conductive cover material comprising about 33% to about 37% nickel, about 30% to about 38% cobalt, about 19% to about 21% chromium, and about 9% to about 10.5% molybdenum.
31. The intravascular device of clause 30, the electrically conductive cover material further comprising about 1% iron.
32. The intravascular device of clause 30 or 21, the electrically conductive cover material further comprising less than 1% of at least one element of a group of elements consisting of: carbon, manganese, silicon, phosphorus, sulfur, titanium, and boron.

What is claimed is:

1. An intravascular device comprising:
a distal section comprising a plurality of electrodes configured to deliver high-voltage pulsed electrical signals between selected ones of the plurality of electrodes; and
a plurality of wires each electrically joined to a respective electrode of the plurality of electrodes, each wire of the plurality of wires comprising:
an electrically conductive core material comprising a first electrical conductivity,
an electrically conductive cover material comprising a second electrical conductivity less than the first electrical conductivity, the electrically conductive cover material circumscribing the electrically conductive core material, and
an insulative jacket circumscribing the electrically conductive cover material such that no electrical arc is generated between the plurality of wires during delivery of the high-voltage pulsed electrical signals between the selected ones of the plurality of electrodes.

2. The intravascular device of claim 1, further comprising an elongated shaft extending along a longitudinal axis and sized to traverse vasculature, each of the wires of the plurality of wires extending through at least a portion of the elongated shaft.

3. The intravascular device of claim 1, the plurality of electrodes being configured to deliver at least 900 Volts between the selected ones of the plurality of electrodes, during delivery of the high-voltage pulsed electrical signals, for irreversible electroporation.

4. The intravascular device of claim 1, the electrically conductive core material comprising either (i) about 12% to about 50% of silver or (ii) about 12% to about 50% of copper.

5. The intravascular device of claim 1, the first electrical conductivity being at least ten times greater than the second electrical conductivity when measured at 20° C.

6. The intravascular device of claim 1,
the electrically conductive core material comprising a first thermal conductivity, and
the electrically conductive cover material comprising a second thermal conductivity less than the first thermal conductivity.

7. The intravascular device of claim 1, each of the wires of the plurality of wires comprising a resistance per length of wire measuring about 1.4 Ω/ft to about 1.8 Ω/ft.

8. The intravascular device of claim 1, each of the electrically conductive cover material of the plurality of wires comprising a diameter measuring between about 0.0031 inches to about 0.0040 inches.

9. An intravascular device comprising:
an elongated shaft extending along a longitudinal axis and sized to traverse vasculature;
a plurality of wires, each of the wires of the plurality of wires extending through at least a portion of the elongated shaft, each wire of the plurality of wires comprising:
a plurality of strands, each strand respectively comprising an electrically conductive core material comprising a first electrical conductivity and an electrically conductive cover material comprising a second electrical conductivity less than the first electrical conductivity, the electrically conductive cover material circumscribing the electrically conductive core material, and
an insulative jacket circumscribing the plurality of strands so that no electrical arc is generated between the plurality of wires when high-voltage pulsed electrical signals are delivered through selected ones of the plurality of wires.

10. The intravascular device of claim 9, further comprising a distal section comprising a plurality of electrodes,
the plurality of wires being each electrically joined to a respective electrode of the plurality of electrodes.

11. The intravascular device of claim 10, the distal section comprising a lasso region upon which the plurality of electrodes are mounted, the lasso region being configured to move between an elongated configuration aligned to the longitudinal axis to a circular shape generally orthogonal to the longitudinal axis.

12. The intravascular device of claim 10, the distal section comprising a plurality of spines upon which the plurality of electrodes are mounted, the spines being configured to move from an elongated configuration aligned to the longitudinal axis such that the spines bow outwardly from the longitudinal axis to form an expanded form.

13. The intravascular device of claim 9, each strand comprising a diameter measuring about 0.002 inches.

14. The intravascular device of claim 9, at least a portion of the wires of the plurality of wires comprising exactly four strands.

15. The intravascular device of claim 9, at least a portion of the strands of the plurality of strands comprising a resistance of about 20 Ω.

16. The intravascular device of claim 9, each of the plurality of wires being configured to withstand bipolar pulses resulting in a voltage difference of about 1,500 volts to about 2,000 volts between two wires in the plurality of wires.

17. The intravascular device of claim 9, the elongated shaft having a diameter of about 8.5 French (2.8 millimeters).

18. The intravascular device of claim 9, the plurality of wires being bundled in an insulative sleeve.

19. The intravascular device of claim 9, each wire of the plurality of wires comprising a diameter measuring about 0.0053 inches.

20. The intravascular device of claim 9, the insulative jacket comprising a wall thickness measuring about 0.0008 inches to about 0.00125 inches.
